SEQUENCE LISTING (14 sequences)

SEQ ID NO: 1 - DNA, 1269 bp, Zea mays
  source 1..1269: mol_type = other DNA
atggggctccttcctaccgaccgcatggacagcctcttctccccgcgctgccagtgggtgaacggccccatcatcgtgggcgcggggccctcgggtctcgccgtggcggcgtgcctgcgcgagcagggcgtcccctacgtcatgctggagcgcgccgactgcatcgcgtcgctgtggcagaggcgcacctacgaccgcctcaagctgcacctgcccaagcagttctgcgagctgccgcgcatgcccttccccgaccactacccggagtaccccacccgccgccagttcatcgactacctcgaggactacgccgccaggttcgagatcaggccggagttcagcaccacggtggtgttggcgcgctacgacgagacgtcgggcctctggcgcgtcaccacctcggcgcccgccaacggtggcgacgtggagtacatcggccgctggctcgtcgtcgccaccggcgagaacgccgaggccgtcgtgcccgacatcccgggtctcggcggcttccacggcaaggtcacccacgtcagcgactacaagtcaggggaggcctacgccgggaagcgcgtgctcgtggtcggctgcggcaactccgggatggaggtgtcgctcgacctctccgaccacggggcccgcccggccatggtcgtgcgcgacgccgtgcacgtcctcccccgtgaggtgctcggcaagtccaccttccagctcgccgtgctcctcaggcgctggctcccgctctggctcgtcgacaagatcatggtcatcctcgcctggctcgtcctgggcaacctcgccaagctcggcctccgacgccccgccgccggcccgctcgagctcaaggagacacacggccgcacacccgtcctcgacaccggcgcgctcgcgcgcatccgtgccggagatatcgccgtcgtccctgccgtgacacgtttcggaaagggcggccaggttgagctcgccgacggacgcactcttgacttcgacgccgtcatcctcgccaccggataccgcagcaacgttccccagtggctcgagagcagcaacgattccttcaacaaggaaggataccccaagacggccttccctcacgggtggaagggtcaatctgggctctacgccgtgggtttcacccggcgtggtctcttgggcgcctccaccgacgccgtgcgcattgccaaggacctcggcaacgtctggagggaggagaccaagcccacgaagagggccggcgcctgccacaggcgctgcatctccgtcgtcttctaa

SEQ ID NO: 2 - protein, 422 aa, Zea mays
MGLLPTDRMDSLFSPRCQWVNGPIIVGAGPSGLAVAACLREQGVPYVMLERADCIASLWQRRTYDRLKLHLPKQFCELPRMPFPDHYPEYPTRRQFIDYLEDYAARFEIRPEFSTTVVLARYDETSGLWRVTTSAPANGGDVEYIGRWLVVATGENAEAVVPDIPGLGGFHGKVTHVSDYKSGEAYAGKRVLVVGCGNSGMEVSLDLSDHGARPAMVVRDAVHVLPREVLGKSTFQLAVLLRRWLPLWLVDKIMVILAWLVLGNLAKLGLRRPAAGPLELKETHGRTPVLDTGALARIRAGDIAVVPAVTRFGKGGQVELADGRTLDFDAVILATGYRSNVPQWLESSNDSFNKEGYPKTAFPHGWKGQSGLYAVGFTRRGLLGASTDAVRIAKDLGNVWREETKPTKRAGACHRRCISVVF

SEQ ID NO: 3 - DNA, 1371 bp, Zea mays
  source 1..1371: mol_type = other DNA
atggggctccttcctaccgaccgcatggacagcctcttctccccgcgctgccagtgggtgaacggccccatcatcgtgggcgcggggccctcgggtctcgccgtggcggcgtgcctgcgcgagcagggcgtcccctacgtcatgctggagcgcgccgactgcatcgcgtcgctgtggcagaggcgcacctacgaccgcctcaagctgcacctgcccaagcagttctgcgagctgccgcgcatgcccttccccgaccactacccggagtaccccacccgccgccagttcatcgactacctcgaggactacgccgccaggttcgagatcaggccggagttcagcaccacggtggtgttggcgcgctacgacgagacgtcgggcctctggcgcgtcaccacctcggcgcccgccaacggtggcgacgtggagtacatcggccgctggctcgtcgtcgccaccggcgagaacgccgaggccgtcgtgcccgacatcccgggtctcggcggcttccacggcaaggtcacccacgtcagcgactacaagtcaggggaggcctacgccgggaagcgcgtgctcgtggtcggctgcggcaactccgggatggaggtgtcgctcgacctctccgaccacggggcccgcccggccatggtcgtgcgcgacgccgtgcacgtcctcccccgtgaggtgctcggcaagtccaccttccagctcgccgtgctcctcaggcgctggctcccgctctggctcgtcgacaagatcatggtcatcctcgcctggctcgtcctgggcaacctcgccaagctcggcctccgacgccccgccgccggcccgctcgagctcaaggagacacacggccgcacacccgtcctcgacaccggcgcgctcgcgcgcatccgtgccggagatatcgccgtcgtccctgccgtgacacgtttcggaaagggcggccaggttgagctcgccgacggacgcactcttgacttcgacgccgtcatcctcgccaccggataccgcagcaacgttccccagtggctcgagcagcaacgattccttcaacaaggaaggataccccaagacggccttccctcacgggtggaagggtcaatctgggctctacgccgtgggtttcacccggcgtggtctcttgggcgcctccaccgacgccgtgcgcattgccaaggacctcggcaacgtctggagggaggagaccaagcccacgaagagggccggcgcctgccacaggcgctgcatctccgtcgtcttctaatccgacccaccagcatgacgacgacgaatgaagctaacttaattactgaagcacacacaaacagacacacagacagatacacttgttcgttcatggtgttttag

SEQ ID NO: 4 - protein, 456 aa, Zea mays
MGLLPTDRMDSLFSPRCQWVNGPIIVGAGPSGLAVAACLREQGVPYVMLERADCIASLWQRRTYDRLKLHLPKQFCELPRMPFPDHYPEYPTRRQFIDYLEDYAARFEIRPEFSTTVVLARYDETSGLWRVTTSAPANGGDVEYIGRWLVVATGENAEAVVPDIPGLGGFHGKVTHVSDYKSGEAYAGKRVLVVGCGNSGMEVSLDLSDHGARPAMVVRDAVHVLPREVLGKSTFQLAVLLRRWLPLWLVDKIMVILAWLVLGNLAKLGLRRPAAGPLELKETHGRTPVLDTGALARIRAGDIAVVPAVTRFGKGGQVELADGRTLDFDAVILATGYRSNVPQWLEQQRFLQQGRIPQDGLPSRVEGSIWALRRGFHPAWSLGRLHRRRAHCQGPRQRLEGGDQAHEEGRRLPQALHLRRLLIRPTSMTTTNEANLITEAHTNRHTDRYTCSFMVF

SEQ ID NO: 5 - DNA, 1266 bp, Zea mays
  source 1..1266: mol_type = other DNA
atggggctcctccctaccgaccgcatggatagcctcttctccccgcgctgcgtgtgggtgaccgggcccatcattgtgggcgcggggccgtcggggctagccgtggcggcgtgcctgcgggagcagggcgtgccgttcgtcgtcctggagcgcgccgactgtatcgcctcgctgtggcaacggcgcacgtacaaccgcctcaagctgcacctacccaagcagttctgccagctcccgcgcatgccgttcccggaagactacccggagtacccgacccgccgccagttcgtcgactacctcgagcgctacgccgccgagttcgagatcaagccggagttcggcaccaccgtgctgtcggcgcgctacgacgagacgtcgggcctctggcgcgtcgtcaccaacggcggagccggcggcgacatggagtacatcgggcgctggctcgtggtcgccacgggcgagaacgcggaggccgtggtgcccgacatcccgggcctcgccggcttcgacggcgaggtgacccacgtgagcgagtacaagtccggcgaggcctacgccggcaagcgcgtgctggtggtcggctgcggcaactccgggatggaggtgtcgctggacctggccgagcacggcgcgcgcccggccatggtggtgcgcgacgccgtccacgtcctcccgcgcgaggtgctgggcacgtccaccttcgggctcgccgtgctgctcatgcgctggctcccgctctggctcgtcgactggctcatggtgctcctggcgtggctcgtcctcggcaacctcgccaggctcggcctccgccgccccgccgccggcccgctccagctcaaggagacgcacggccgcacaccagtcctcgactacggcgcgctcgcgcgcatccgcgccggcgacatcaccgtcgtccctgcggtgacacggttcgccggcaagggcggacaggttgaggtcgccgacggccgcacgctcggcttcgacgccgtcatcctcgccaccggatatcgcagcaacgtgccgcagtggctccagggcaacgatttcttcaacaaggacgggtaccccaagacggcgttcccgcacgggtggaagggcgagagtgggctgtacgccgtgggcttcacccggcgcggcctctccggcgcctccgccgacgccgtgcgcatcgccaaggacctcgggaacgtctggagggaggagaccaagcccaccaagagggccggcgcctgccacaggcgctgcatctcggtcgtcttctaa

SEQ ID NO: 6 - protein, 421 aa, Zea mays
MGLLPTDRMDSLFSPRCVWVTGPIIVGAGPSGLAVAACLREQGVPFVVLERADCIASLWQRRTYNRLKLHLPKQFCQLPRMPFPEDYPEYPTRRQFVDYLERYAAEFEIKPEFGTTVLSARYDETSGLWRVVTNGGAGGDMEYIGRWLVVATGENAEAVVPDIPGLAGFDGEVTHVSEYKSGEAYAGKRVLVVGCGNSGMEVSLDLAEHGARPAMVVRDAVHVLPREVLGTSTFGLAVLLMRWLPLWLVDWLMVLLAWLVLGNLARLGLRRPAAGPLQLKETHGRTPVLDYGALARIRAGDITVVPAVTRFAGKGGQVEVADGRTLGFDAVILATGYRSNVPQWLQGNDFFNKDGYPKTAFPHGWKGESGLYAVGFTRRGLSGASADAVRIAKDLGNVWREETKPTKRAGACHRRCISVVF

SEQ ID NO: 7 - DNA, 3000 bp, Zea mays
  source 1..3000: mol_type = genomic DNA
ggcataggatactacaggcaggtgaacatggtccttggtaatcttgttcgtctccactggcctggtcttgtgacttttcctagtggcgagtctgtccccgccaccacttgggagcattatcgctatggtgtctgtagaacgtttggcaacacacaggcactagtttgggatgcattcttggtatgaattgtttttactaatttagttatcccatatatggttgcttgtacgataacactatttgtttgcagaaacggtacaagttgccggaagatggatcatatgatatgaacgctcgttacgtgtttgagtataacatgaacgatgtcattgcagatgcaaagtactatgcacgaattcaggctataaaggcatggtacagagcaagtgctgatgatcggcccatgccgaataccaaggcggagtggtcatcaatttaccttacggaggagcaataccttgaggtaaacaagttgttgcatctcatatcgttttttgctgattcttgttttgggtacatgttaatccatgtatttgcttgattaaaaaaaattcatataggtgtcggtgccgtggatggccacccgataagagggttatcgggccttgtgcagatggtggcttcccctgagtttcgtgccatttctgaaaggaacagaggaaatcgtggactcagtcgttccataactacggcggcgatggacttgtgcgcttggctaagcgattggtatgtcacagtatgttgtaacttcgaattacataaaaatgtgtcattctaacttgtatgtacaggaagtcaaatccggccgtacgcccatggatgtggaggtgtatatgcaagggcataggggttctgatcctcagaatcctgatgttttatgcactcagacggccaccgaccgtctagtgagtttttggtactctattatgtgtttgtgcatctaattggtgatgcacttatatttggtatgtttgcctccaggcttcggacgggcaggagatggttcaatgccatggacatgagtacgattggaggagccagccaatcgaccctcaggcagcatatgctagcgcagaaggacaagctcatggacggtgggagtatttgatttggatttcaaaattatcctcatttgcttgcgattgaactaagtacatgatttactgtactaagtgcatggttcactcttgtaggtcgggtatttttgattctacgattaattctagggagctgagatgccgtgaacgacagtccacatcctcgtcttcgcagacgtcccattctcgatcatccgcataagagatagagcttgcagtgttgcgtcaacaggcagagtatcatcaatcagttttgagggaacaaatggagtaccagagacaacaagctgagtaccagaagaaaaaggacgggtattattcaaacctccaggcccataatcaagctcttctctcggtaagttgaagtaacattttgttcataacaactaaaaacacatgatgtgtatcttattttctcaacaatggcttgtatataatttgtagcaactgacacaacaagcgagcgtcctgatgccgacatatgggatgccgcctccggactttgcactgccgatgacaatgctgtcgcctccacctccgtctctgtcacaattccctatggtatgtacacatatgcgtgggtgacatgttcatagatgtcttatgtgttaaaatgaaaaacttagtggttaatatttcacgcatatgtgttatagggatttcagacaccacccgctttagttgccgcacctggagatgggtctggccaagacgacgcatcgcattcttgggtgaacaaccttttcaacacccagagtccacccggaggaagtggctataacttgaaccaaccaggtgatggatatgagtgatagttcatcaaatgtgtttgatttagaactgtcactgttcatgtcaaagactatgtgtcgaactatgtcagtttttaatttggaactatatgttgatgtaaaagacttgttgcaactatttgggactatatgtttgtgaatgtggtgctgttaaatttgtgaatgtgaatacttatgtgaattgatgtttgtgaatgtaaatatgtggttgtatatgaaatctgtgtatatgtgtatgcaatatgtgattttttctgtaaatatcaaaaatttataaaaacatgattttttttgcaattgctgaaattcgttatgtccgacggcctacgctaggccgtcggacataagggttgtaggccccacaagtagtcgttaaaacggttgacgagagttatcttcgacggcctggcgtggctgtcgaaggtagcctatgtccgacggtcaccgtcagaaataacactatttccgaccagttacggccgagggctgggtaccgtcggagataaggatttggccgtcggaaatagactatttccgacggtttaacctttatatccaacggttttggccgtgttggacattctatattttaccgtagtgacgataattagatatgctttcgatctccatgtaatcttccccggcctgccatgcatgcccactactttatttgcctgctcgttcgttcgttgtagctactgcattgccgtgatcgttcattatcatcatgcagtcatgcagatgccaagggtgatgaggccgcgccgcgcgcagtgcctcccctcgatctcctctccaccacctaaagcagcagcaaccgcaccactactccctccgtttcctctctacttcacatgcacaccaactcgtctctccctctccctatataacaccccctagtccagcccccaaaccaccaacacaacaacatctcttccagctcccaccagcaactacaagagaacatcacatcctagctagctactagctcgagctctcatcagaagaacatactgatcatcgatcaccagtcaccaccaccacctaaggctctcagacgacctcagagattgaaagccacacatcagcc

SEQ ID NO: 8 - DNA, 3000 bp, Zea mays
  source 1..3000: mol_type = genomic DNA
ctttgtggtgtggccgaaccttgggataaatcttgtgtcttgtgctcttgcttggattcgtggttgtgttttagttggcaagaaaggcatatagagttgttcttcgtgtggatctcgtggtggattcactcttgtatttctcaccatccacacttagtttatcccttgtattttcgttctaaaggaatctctcaatgtatttccgtgtagttcgaagcctagatttactctagctgttggacaggttcagagtggttcaactcgcctctatatcagttgaactggcctacaccagttgaactgttgatttgacattatcttgaagtttgtggtgataatttttaggtttagcctattcaccccctcctctaggctattttcaaggtctaaagcttggattgacttcaaagaggtaaaaatggtgaacggtaagagggtaagggtctttgccatctacatccactgtaagcaaacattttctactaaatcatctggttgcactggacatttgctacgccatattgatacttgtgctgctaaggaagaaaaggaacaatctggtagaattcgatctgttcttaaatataatcccgttggttcacttatgatgctgctatatctgagttatcatcatatcttgatagtgatactattacccagtttgactcttatctcaatcttctaaactagtggcaacggcacaagttgacctatcatgtgctttctatactttctaaagatgttctaactgtgtctgcttccactttatcatcagagtctacttttagtttagctggcaggttgcttgaggaccagagatggctaactcctgatacggttgaagtcttgtcctggataaagaactgggagctcgttgacttgcacagtcagcacacggtggagaaagaggccaaggaacttgaaggtgtttttcaaacaatgtacctagatgaaaatgccaacaatgagaacagagggcaagaaagaagaggtggaggtggaactggtggtagatgttgagcaattgagagtcgttgttattgcttcttaattctgttgctattactactctattataacttgtgatgaactattaaactcgactggacttgaacattaaacctaaaggagctggttgtactcttttcctttctagggttttcttacgaggtatgaatttttacctaggatggtttttaacgaggcagcattgcattaaggcaccagactccagtagtattttgtttgcatcaatctttgtctatctctctgtaagccgtgattgagtctatgtcactgtgtttgatagtttgagtctaatcatgagtttgtgtgaactgtttctgctatatctaagaattttatagtttgtgactttgtgtagaattgttgtcgttatatttgagtatttcatagtttgcgactttgtgtgcaattcggctatatttcagtatttcataatatgtggtttacaatttgatttatgttgaattttgattttttttttgcgtgtttcgggccaggccgcaagcacgacacgacacaatttccattttttcggtgctatgtttgggccggctcaacatactgcaggctggcgcggcccgacgcgaacctagcattgtgttgtgcatggcactcgggtaactatgtcatgtctgatcgtgtccgtgccgtgctggcgtggcacgcacaatggacatgtatagatatcacagtcgtagcatgcatacatggaatttccgacgacaaataatctctaagccgtcggacataagaaggttgtcgaaaatgtgtaattttactgtagtggcaaatggaatagctgaggaatgggggcaaagtgcacctggattaacccaaccaactagccgtggagattaattaattaattaatctgaaactaattttgactctcgcatgcaacgacgtggatgtggatctcaataatagtggagcgcgatcgaggaggcgcggcgcgcgcgcgtgcctgactgcgggaagcgcgagcggcgcgggcggccggccggccggccggtcgatcggtccaacactcttcaacccgctcgtcaccgttggtgatggtggtggcgatggcgtggaccgtggacgcatgcatagcatcgaggatgcagagggaatccacgtacgccaaacaatggcagcggcggcgcacaccaccctccgccccactgggcgaagggcatccctgcatgtgtagtcagtgagtgagtcggtcgctcgagctagccaccctcatcctcagaatctctcataaaacgagataaaatctgcatcgaattttctcattccacaccggcccagaacgagaagaggaggggattttaaaactcgaattccggaagaaaaaaaggggacttttagatcctctcgatctccatgtaatcttccaccattttgtttgtctgttcgttcgttattgtttgtagctactgcgtgcatggtgcatgccaatattcttcctctgctgccctgcctctctctctcatcatgcagagcagatgcagactccatcgatctccattccgtccccaccagcatgcagcaacagcaccactgctttctctctcctcgtcttcctctctctctctctctctctctctctctctctctctctctctctctctctctctctctctctctctctctctccttcgaattcacatgcctacagttgttcggcacacacaatgctgtccggcgtctccctctccctccatcctgattcctgacccaccctccctccttcccttctctatataaccaccactagtccaacccccaaaccaccaacacaacaacatctcctccgttctcctagctccagcaacaaacaggacacaacaccttctcctcctcattcttctaccatagctagctagctagcttcacatcagcagctacatactcaccgatagatacagacctcagagcatcacactacagagtacagacctagctcagagcacacatccagcc

SEQ ID NO: 9 - DNA, 2050 bp, Zea mays
  source 1..2050: mol_type = genomic DNA
accaacacaacaacatctcttccagctcccaccagcaactacaagagaacatcacatcctagctagctactagctcgagctctcatcagaagaacatactgatcatcgatcaccagtcaccaccaccacctaaggctctcagacgacctcagagattgaaagccacacatcagccatggggctccttcctaccgaccgcatggacagcctcttctccccgcgctgccagtgggtgaacggccccatcatcgtgggcgcggggccctcgggtctcgccgtggcggcgtgcctgcgcgagcagggcgtcccctacgtcatgctggagcgcgccgactgcatcgcgtcgctgtggcagaggcgcacctacgaccgcctcaagctgcacctgcccaagcagttctgcgagctgccgcgcatgcccttccccgaccactacccggagtaccccacccgccgccagttcatcgactacctcgaggactacgccgccaggttcgagatcaggccggagttcagcaccacggtggtgttggcgcgctacgacgagacgtcgggcctctggcgcgtcaccacctcggcgcccgccaacggtggcgacgtggagtacatcggccgctggctcgtcgtcgccaccggcgagaacgccgaggccgtcgtgcccgacatcccgggtctcggcggcttccacggcaaggtcacccacgtcagcgactacaagtcaggggaggcctacgccgggaagcgcgtgctcgtggtcggctgcggcaactccgggatggaggtgtcgctcgacctctccgaccacggggcccgcccggccatggtcgtgcgcgacgccgtgcacgtcctcccccgtgaggtgctcggcaagtccaccttccagctcgccgtgctcctcaggcgctggctcccgctctggctcgtcgacaagatcatggtcatcctcgcctggctcgtcctgggcaacctcgccaagctcggcctccgacgccccgccgccggcccgctcgagctcaaggagacacacggccgcacacccgtcctcgacaccggcgcgctcgcgcgcatccgtgccggagatatcgccgtcgtccctgccgtgacacgtttcggaaagggcggccaggttgagctcgccgacggacgcactcttgacttcgacgccgtcatcctcgccaccggataccgcagcaacgttccccagtggctcgaggtaaatccgtaaatccaataattcgaatgccaataatatccaagtatgtactactgttccattgctagaacattgttgatctgatcaatctccatctccaattaattctttcagagcagcaacgattccttcaacaaggaaggataccccaagacggccttccctcacgggtggaagggtcaatctgggctctacgccgtgggtttcacccggcgtggtctcttgggcgcctccaccgacgccgtgcgcattgccaaggacctcggcaacgtctggagggaggagaccaagcccacgaagagggccggcgcctgccacaggcgctgcatctccgtcgtcttctaatccgacccaccagcatgacgacgacgaatgaagctaacttaattactgaagcacacacaaacagacacacagacagatacacttgttcgttcatggtgttttagttagtcagtccaatccccttggttcgttgttcattaatttgttctttgcctgccaggtacattagtagtaatacggtgagagtgagttgagtaaaaaaaaaaacatggagccctcccatcagtagggaagaagtgagtgagccctagaggggattttttttactgcagctatggccgctagcttggtgtatagtagcaacctgttgatggaagaagaggctctctttatttttcattgacctaccagcatgtaaattatttaatttatttgttgttctatacacaccaccggtgtattagttactctactgcagtttgtagcttgtagtagtagccagttttagtactgtatggttatagtgatgaagaggtatatcacatcttgttg

SEQ ID NO: 10 - DNA, 2063 bp, Zea mays
  source 1..2063: mol_type = genomic DNA
accaacacaacaacatctcctccgttctcctagctccagcaacaaacaggacacaacaccttctcctcctcattcttctaccatagctagctagctagcttcacatcagcagctacatactcaccgatagatacagacctcagagcatcacactacagagtacagacctagctcagagcacacatccagccatggggctcctccctaccgaccgcatggatagcctcttctccccgcgctgcgtgtgggtgaccgggcccatcattgtgggcgcggggccgtcggggctagccgtggcggcgtgcctgcgggagcagggcgtgccgttcgtcgtcctggagcgcgccgactgtatcgcctcgctgtggcaacggcgcacgtacaaccgcctcaagctgcacctacccaagcagttctgccagctcccgcgcatgccgttcccggaagactacccggagtacccgacccgccgccagttcgtcgactacctcgagcgctacgccgccgagttcgagatcaagccggagttcggcaccaccgtgctgtcggcgcgctacgacgagacgtcgggcctctggcgcgtcgtcaccaacggcggagccggcggcgacatggagtacatcgggcgctggctcgtggtcgccacgggcgagaacgcggaggccgtggtgcccgacatcccgggcctcgccggcttcgacggcgaggtgacccacgtgagcgagtacaagtccggcgaggcctacgccggcaagcgcgtgctggtggtcggctgcggcaactccgggatggaggtgtcgctggacctggccgagcacggcgcgcgcccggccatggtggtgcgcgacgccgtccacgtcctcccgcgcgaggtgctgggcacgtccaccttcgggctcgccgtgctgctcatgcgctggctcccgctctggctcgtcgactggctcatggtgctcctggcgtggctcgtcctcggcaacctcgccaggctcggcctccgccgccccgccgccggcccgctccagctcaaggagacgcacggccgcacaccagtcctcgactacggcgcgctcgcgcgcatccgcgccggcgacatcaccgtcgtccctgcggtgacacggttcgccggcaagggcggacaggttgaggtcgccgacggccgcacgctcggcttcgacgccgtcatcctcgccaccggatatcgcagcaacgtgccgcagtggctccaggtaaattcacagtccaagaattgaaatccatcaaatataatgctactgttccattgcaaacaagtcaagagcattcccttcgttgatctcatcaactaatctcctccgtctccaaaaatgtgttctttcagggcaacgatttcttcaacaaggacgggtaccccaagacggcgttcccgcacgggtggaagggcgagagtgggctgtacgccgtgggcttcacccggcgcggcctctccggcgcctccgccgacgccgtgcgcatcgccaaggacctcgggaacgtctggagggaggagaccaagcccaccaagagggccggcgcctgccacaggcgctgcatctcggtcgtcttctaatccgatcggccacacctgggagcatgcatgacgatgattgaagctaacttttctgaagcgcacacacagctgacagacagttgttcgttgatggtgttttagttagtctcacagcacgtccaatccccttggtttgttgttgatttgttctttgagatcgaggtacatcagtagtgagtaatatacgggtaaaaacagtgagagtgagttgagtaaaagaaaagaaaagaatggagccctcccatcagtaggggaagtgagtgagccctagcctaggggatttttactgcagctatggccgctagcttgctgtatagtagcaacctgttgatggaggaagaggctctctctttatgttttatttatgtttcgtcgacctagcagctagctagctagcatgttcatttctttcttctgttgaatgtatatggctactgttaattagtagtgtaatttgaaagtccatcgtttcgtt

SEQ ID NO: 11 - DNA, 1378 bp, Zea mays
  source 1..1378: mol_type = genomic DNA
atggggctccttcctaccgaccgcatggacagcctcttctccccgcgctgccagtgggtgaacggccccatcatcgtgggcgcggggccctcgggtctcgccgtggcggcgtgcctgcgcgagcagggcgtcccctacgtcatgctggagcgcgccgactgcatcgcgtcgctgtggcagaggcgcacctacgaccgcctcaagctgcacctgcccaagcagttctgcgagctgccgcgcatgcccttccccgaccactacccggagtaccccacccgccgccagttcatcgactacctcgaggactacgccgccaggttcgagatcaggccggagttcagcaccacggtggtgttggcgcgctacgacgagacgtcgggcctctggcgcgtcaccacctcggcgcccgccaacggtggcgacgtggagtacatcggccgctggctcgtcgtcgccaccggcgagaacgccgaggccgtcgtgcccgacatcccgggtctcggcggcttccacggcaaggtcacccacgtcagcgactacaagtcaggggaggcctacgccgggaagcgcgtgctcgtggtcggctgcggcaactccgggatggaggtgtcgctcgacctctccgaccacggggcccgcccggccatggtcgtgcgcgacgccgtgcacgtcctcccccgtgaggtgctcggcaagtccaccttccagctcgccgtgctcctcaggcgctggctcccgctctggctcgtcgacaagatcatggtcatcctcgcctggctcgtcctgggcaacctcgccaagctcggcctccgacgccccgccgccggcccgctcgagctcaaggagacacacggccgcacacccgtcctcgacaccggcgcgctcgcgcgcatccgtgccggagatatcgccgtcgtccctgccgtgacacgtttcggaaagggcggccaggttgagctcgccgacggacgcactcttgacttcgacgccgtcatcctcgccaccggataccgcagcaacgttccccagtggctcgaggtaaatccgtaaatccaataattcgaatgccaataatatccaagtatgtactactgttccattgctagaacattgttgatctgatcaatctccatctccaattaattctttcagagcagcaacgattccttcaacaaggaaggataccccaagacggccttccctcacgggtggaagggtcaatctgggctctacgccgtgggtttcacccggcgtggtctcttgggcgcctccaccgacgccgtgcgccaaggacctcggcaacgtctggagggaggagaccaagcccacgaagagggccggcgcctgccacaggcgctgcatctccgtcgtcttctaa

SEQ ID NO: 12 - DNA, 1384 bp, Zea mays
  source 1..1384: mol_type = genomic DNA
atggggctccttcctaccgaccgcatggacagcctcttctccccgcgctgccagtgggtgaacggccccatcatcgtgggcgcggggccctcgggtctcgccgtggcggcgtgcctgcgcgagcagggcgtcccctacgtcatgctggagcgcgccgactgcatcgcgtcgctgtggcagaggcgcacctacgaccgcctcaagctgcacctgcccaagcagttctgcgagctgccgcgcatgcccttccccgaccactacccggagtaccccacccgccgccagttcatcgactacctcgaggactacgccgccaggttcgagatcaggccggagttcagcaccacggtggtgttggcgcgctacgacgagacgtcgggcctctggcgcgtcaccacctcggcgcccgccaacggtggcgacgtggagtacatcggccgctggctcgtcgtcgccaccggcgagaacgccgaggccgtcgtgcccgacatcccgggtctcggcggcttccacggcaaggtcacccacgtcagcgactacaagtcaggggaggcctacgccgggaagcgcgtgctcgtggtcggctgcggcaactccgggatggaggtgtcgctcgacctctccgaccacggggcccgcccggccatggtcgtgcgcgacgccgtgcacgtcctcccccgtgaggtgctcggcaagtccaccttccagctcgccgtgctcctcaggcgctggctcccgctctggctcgtcgacaagatcatggtcatcctcgcctggctcgtcctgggcaacctcgccaagctcggcctccgacgccccgccgccggcccgctcgagctcaaggagacacacggccgcacacccgtcctcgacaccggcgcgctcgcgcgcatccgtgccggagatatcgccgtcgtccctgccgtgacacgtttcggaaagggcggccaggttgagctcgccgacggacgcactcttgacttcgacgccgtcatcctcgccaccggataccgcagcaacgttccccagtggctcgaggtaaatccgtaaatccaataattcgaatgccaataatatccaagtatgtactactgttccattgctagaacattgttgatctgatcaatctccatctccaattaattctttcagagcagcaacgattccttcaacaaggaaggataccccaagacggccttccctcacgggtggaagggtcaatctgggctctacgccgtgggtttcacccggcgtggtctcttgggcgcctccaccgacgccgtgcagcattgccaaggacctcggcaacgtctggagggaggagaccaagcccacgaagagggccggcgcctgccacaggcgctgcatctccgtcgtcttctaa

SEQ ID NO: 13 - DNA, 1395 bp, Zea mays
  source 1..1395: mol_type = genomic DNA
atggggctcctccctaccgaccgcatggatagcctcttctccccgcgctgcgtgtgggtgaccgggcccatcattgtgggcgcggggccgtcggggctagccgtggcggcgtgcctgcgggagcagggcgtgccgttcgtcgtcctggagcgcgccgactgtatcgcctcgctgtggcaacggcgcacgtacaaccgcctcaagctgcacctacccaagcagttctgccagctcccgcgcatgccgttcccggagactacccggagtacccgacccgccgccagttcgtcgactacctcgagcgctacgccgccgagttcgagatcaagccggagttcggcaccaccgtgctgtcggcgcgctacgacgagacgtcgggcctctggcgcgtcgtcaccaacggcggagccggcggcgacatggagtacatcgggcgctggctcgtggtcgccacgggcgagaacgcggaggccgtggtgcccgacatcccgggcctcgccggcttcgacggcgaggtgacccacgtgagcgagtacaagtccggcgaggcctacgccggcaagcgcgtgctggtggtcggctgcggcaactccgggatggaggtgtcgctggacctggccgagcacggcgcgcgcccggccatggtggtgcgcgacgccgtccacgtcctcccgcgcgaggtgctgggcacgtccaccttcgggctcgccgtgctgctcatgcgctggctcccgctctggctcgtcgactggctcatggtgctcctggcgtggctcgtcctcggcaacctcgccaggctcggcctccgccgccccgccgccggcccgctccagctcaaggagacgcacggccgcacaccagtcctcgactacggcgcgctcgcgcgcatccgcgccggcgacatcaccgtcgtccctgcggtgacacggttcgccgcaagggcggacaggttgaggtcgccgacggccgcacgctcggcttcgacgccgtcatcctcgccaccggatatcgcagcaacgtgccgcagtggctccaggtaaattcacagtccaagaattgaaatccatcaaatataatgctactgttccattgcaaacaagtcaagagcattcccttcgttgatctcatcaactaatctcctccgtctccaaaaatgtgttctttcagggcaacgatttcttcaacaaggacgggtaccccaagacggcgttcccgcacgggtggaagggcgagagtgggctgtacgccgtgggcttcacccggcgcggcctctccggcgcctccgccgacgccgtgcgcatcgccaaggacctcgggaacgtctggagggaggagaccaagcccaccaagagggccggcgcctgccacaggcgctgcatctcggtcgtcttctaa

SEQ ID NO: 14 - DNA, 1380 bp, Zea mays
  source 1..1380: mol_type = genomic DNA
atggggctcctccctaccgaccgcatggatagcctcttctccccgcgctgcgtgtgggtgaccgggcccatcattgtgggcgcggggccgtcggggctagccgtggcggcgtgcctgcgggagcagggcgtgccgttcgtcgtcctggagcgcgccgactgtatcgcctcgctgtggcaacggcgcacgtacaaccgcctcaagctgcacctacccaagcagttctgccagctcccgcgcatgccgttcccgtacccgacccgccgccagttcgtcgactacctcgagcgctacgccgccgagttcgagatcaagccggagttcggcaccaccgtgctgtcggcgcgctacgacgagacgtcgggcctctggcgcgtcgtcaccaacggcggagccggcggcgacatggagtacatcgggcgctggctcgtggtcgccacgggcgagaacgcggaggccgtggtgcccgacatcccgggcctcgccggcttcgacggcgaggtgacccacgtgagcgagtacaagtccggcgaggcctacgccggcaagcgcgtgctggtggtcggctgcggcaactccgggatggaggtgtcgctggacctggccgagcacggcgcgcgcccggccatggtggtgcgcgacgccgtccacgtcctcccgcgcgaggtgctgggcacgtccaccttcgggctcgccgtgctgctcatgcgctggctcccgctctggctcgtcgactggctcatggtgctcctggcgtggctcgtcctcggcaacctcgccaggctcggcctccgccgccccgccgccggcccgctccagctcaaggagacgcacggccgcacaccagtcctcgactacggcgcgctcgcgcgcatccgcgccggcgacatcaccgtcgtccctgcggtgacacggttcgcgcaagggcggacaggttgaggtcgccgacggccgcacgctcggcttcgacgccgtcatcctcgccaccggatatcgcagcaacgtgccgcagtggctccaggtaaattcacagtccaagaattgaaatccatcaaatataatgctactgttccattgcaaacaagtcaagagcattcccttcgttgatctcatcaactaatctcctccgtctccaaaaatgtgttctttcagggcaacgatttcttcaacaaggacgggtaccccaagacggcgttcccgcacgggtggaagggcgagagtgggctgtacgccgtgggcttcacccggcgcggcctctccggcgcctccgccgacgccgtgcgcatcgccaaggacctcgggaacgtctggagggaggagaccaagcccaccaagagggccggcgcctgccacaggcgctgcatctcggtcgtcttctaa

(19) United States
(12) Patent Application Publication
(10) Pub. No.: US 2025/0034585 A1
(43) Pub. Date: Jan. 30, 2025

(54) GENES FOR REGULATING ROOT GROWTH ANGLE AND LOGING RESISTANCE IN MAIZE AND APPLICATIONS THEREOF

(71) Applicants: BIOTECHNOLOGY RESEARCH INSTITUTE OF CHINESE ACADEMY OF AGRICULTURAL SCIENCES, Beijing (CN); SOUTH CHINA AGRICULTURAL UNIVERSITY, Guangzhou (CN)

(72) Inventors: Haiyang WANG, Guangzhou (CN); Baobao WANG, Beijing (CN); Zhigang ZHENG, Guangzhou (CN); Chuyun ZHUO, Guangzhou (CN); Dexin KONG, Guangzhou (CN); Miaoyun XU, Beijing (CN); Yurong XIE, Beijing (CN)

(73) Assignees: BIOTECHNOLOGY RESEARCH INSTITUTE OF CHINESE ACADEMY OF AGRICULTURAL SCIENCES, Beijing (CN); SOUTH CHINA AGRICULTURAL UNIVERSITY, Guangzhou (CN)

(21) Appl. No.: 18/914,003

(22) Filed: Oct. 11, 2024

Related U.S. Application Data
(63) Continuation of application No. PCT/CN2022/130494, filed on Nov. 8, 2022.

(30) Foreign Application Priority Data
Apr. 22, 2022 (CN) 202210423670.9

(51) Int. Cl. C12N 15/82; C12N 9/22

(52) U.S. Cl. CPC C12N 15/8271; C12N 9/22; C12N 15/8201; C12N 15/8218; C12N 2310/20

(57) ABSTRACT

Provided are ZmYUC2 and ZmYUC4 genes for regulating and controlling an included angle and the lodging resistance of a maize root system, and use thereof. The ZmYUC2 and ZmYUC4 genes can specifically regulate and control local auxin synthesis of a root tip, and can regulate and control the included angle of the maize root system without an adverse effect on the remaining agronomic traits, so that the ZmYUC2 and ZmYUC4 genes can be applied to a lodging-resistant breeding variety.

### RELATED APPLICATIONS

The present application claims priority to Chinese Patent Application No. CN202210423670.9, filed with the China National Intellectual Property Administration on Apr. 22, 2022, and entitled “GENES FOR REGULATING ROOT GROWTH ANGLE AND LOGING RESISTANCE IN MAIZE AND APPLICATIONS THEREOF”, the disclosure of which is incorporated herein by reference in its entirety.

The present patent document is a continuation of PCT Application Serial No. PCT/CN2022/130494, filed Nov. 8, 2022, designating the United States and published in English, which is hereby incorporated by reference.

### BACKGROUND

### 1. Technical Field Text

The present application belongs to the field of biological gene technologies, and in particular relates to a gene for regulating a root growth angle and lodging resistance in maize.

### 2. Background Information

Maize, an important agricultural crop with the highest production worldwide, serves as a crucial source for food, feed, and industrial raw materials. Its stable and adequate supply is essential for global food security. As maize lodging has become more and more severe due to climate deterioration, various environmental stressors and disasters, extensive use of nitrogen fertilizers, and dense planting practices in recent years, it is considered as the main limiting factor for high and stable yields of maize.

Maize lodging refers to the phenomenon where maize roots or stems bend or break due to external forces. The harms of lodging mainly include: 1) disrupting the spatial order of leaves to cause plant collisions that damage leaf tissues, leading to reduced photosynthetic efficiency and compromised yield; 2) damaging the root and stem transport systems to affect the transport of nutrients, water, and photosynthetic products, resulting in yield reduction; 3) inducing ear sprouting to increase the occurrence of ear diseases, leading to compromised maize quality; 4) causing disorderly plant arrangement, which significantly increases the difficulty and cost of harvesting. Existing statistics have shown that lodging can reduce maize yield by 15-50%, and in severe cases, lead to complete crop failure. For every 1% increase in lodging rate, maize yield can decrease by approximately 108 kg/hm. Surveys have also shown that among all traits including yield, lodging resistance is the most concerning trait to farmers and is the primary factor considered when selecting varieties. Therefore, improving lodging resistance is the primary breeding objective in developing new maize varieties.

Maize lodging is typically categorized into two types: root lodging and stem lodging. Root lodging occurs almost throughout the entire maize growth period and in a more extensive range, representing the primary lodging disaster affecting maize production. Studies have shown that the configuration of the root system is the main factor affecting maize root lodging. The root system is the primary organ for plant anchorage and underground nutrient uptake. Maize root systems mainly include radicle and nodal root systems. The radicle system is mainly composed of primary roots and seminal roots, reaching its maximum during the V2 stage (a stage with two leaves with visible leaf collars), serving as the primary organ for anchoring, water uptake, and underground nutrient acquisition in maize seedlings. The nodal root system mainly refers to roots growing on maize stem nodes, including crown roots growing underground on stem nodes and brace roots growing aboveground on stem nodes. The brace roots can “grip” the soil to form a conical structure, effectively keeping maize plants upright. In general, the uppermost two layers of brace roots (typically growing on the 6th-7th nodes of the stem) can account for 50% of the total nodal roots, representing the primary functional root system of maize. Therefore, the brace roots are the main organ affecting the root lodging resistance and nutrient absorption capacity of maize.

Due to the complexity of the maize root system, difficulties in phenotype measurement, high costs, susceptibility to environmental influences and other factors, genetic studies on maize nodal root development and configuration regulation as well as cloning and functional studies of lodging resistance genes, are still insufficient in China and even the world.

### BRIEF SUMMARY

All references mentioned herein are incorporated herein by reference. Unless otherwise indicated, all technical and scientific terms used herein have the same meanings as generally understood by those of ordinary skill in the art to which the present invention belongs. Unless otherwise indicated, the techniques used or mentioned herein are standard techniques generally known to those of ordinary skill in the art. The materials, methods and examples are for illustrative purposes, rather than limiting.

The objective of embodiments of the present application is to provide genes ZmYUC2 and ZmYUC4 related to regulating of a brace root angle and lodging resistance in maize, applications thereof, and a method for enhancing lodging resistance in maize.

An embodiment of the present application provides a gene for regulating a root growth angle and lodging resistance in maize, wherein said gene when mutated in a plant is capable of increasing a brace root growth angle and thus enhancing the lodging resistance in maize, and the nucleotide sequence of said gene is selected from one of the following sequences:
(a) a polynucleotide sequence as set forth in SEQ ID No: 1, 3, 5, 9, or 10;
(b) a polynucleotide sequence as set forth in SEQ ID No: 2, 4, or 6, as an encoded amino acid sequence of said gene;
(c) a polynucleotide sequence that hybridizes with the polynucleotide sequence in (a) or (b) under stringent hybridization conditions, wherein the mutation of the said polynucleotide sequence endows the maize with the increased brace root growth angle and lodging resistance;
(d) a polynucleotide sequence that has at least 90%, 95%, 98% or greater identity to the polynucleotide sequence as set forth in any of (a)-(c), wherein the mutation of the polynucleotide sequence endows the maize with increased brace root growth angle and lodging resistance; or
(e) a polynucleotide sequence that is complementary to the sequence as set forth in any of (a)-(d).

In the embodiments of the present application, the term “increased brace root angle” or “increased brace root growth angle” refers to the same concept. It denotes an increase in the angle between the brace roots and the maize stem to form a “conical shape”, which enhances the lodging resistance of maize plants.

The genes provided in the embodiments of the present application further comprise a homologous gene having at least 80%, 85%, 90%, 95%, 98%, or 99% sequence identity to the nucleotide sequences of the lodging-related gene disclosed in the embodiments of the present invention, or a homologous gene having at least 90%, 95%, or 98% sequence identity to the amino acid sequences of the lodging-related gene disclosed in the embodiments of the present invention. When mutated in maize, these homologous genes increase the brace root growth angle, thereby enhancing the plant's lodging resistance, and can be isolated from any maize variety.

Those skilled in the art should be aware that single nucleotide polymorphisms (SNPs) exist in the same gene among different varieties of the same plant species. That is, the nucleotide sequence of the same gene often has individual base differences, but there are many varieties of the same crop, and the inventor cannot list them one by one. The embodiments of the present application only provide sequences of representative varieties in maize. Therefore, those skilled in the art should be aware that nucleotide sequences from different variety sources that have SNP with the genes and their sequences claimed in the present invention should also fall within the scope of the present invention.

The percentage of sequence identity described in the present application may be obtained using well-known bioinformatics algorithms, including Myers and Miller algorithm, Needleman-Wunsch global alignment method, Smith-Waterman local alignment method, Pearson and Lipman identity search method, and Karlin and Altschul algorithm, which are well-known to those skilled in the art.

An embodiment of the present application provides an expression cassette, a recombinant vector, or a cell, wherein said expression cassette, said recombinant vector or said cell comprises part or all of a gene sequence for regulating a root growth angle and lodging resistance in maize, and said gene sequence is selected from one of the following polynucleotide sequences:
(a) a polynucleotide sequence as set forth in SEQ ID No: 1, 3, 5, 9, or 10;
(b) a polynucleotide sequence as set forth in SEQ ID No: 2, 4, or 6, as an encoded amino acid sequence of said gene;
(c) a polynucleotide sequence that hybridizes with the polynucleotide sequence in (a) or (b) under stringent hybridization conditions, wherein the mutation of said polynucleotide sequence endows the maize with the increased root growth angle and lodging resistance;
(d) a polynucleotide sequence that has at least 95% or greater identity to the polynucleotide sequence as set forth in any of (a)-(c);
(e) a sequence that has at least 300 or 500 continuous polynucleotide sequences from the polynucleotide sequence as set forth in any of (a)-(d); or
a polynucleotide sequence that is complementary to the sequence as set forth in any of (a)-(e).

An embodiment of the present application further provides a gene mutant sequence that is derived from a mutation of a genome nucleotide sequence or promoter sequence, and generates a phenotype of an increased brace root angle and lodging resistance in maize, wherein said gene nucleotide sequence is selected from one of the following sequences:
(a) a polynucleotide sequence as set forth in SEQ ID No: 1, 3, 5, 9, or 10;
(b) a polynucleotide sequence as set forth in SEQ ID No: 2, 4, or 6, as an encoded amino acid sequence of said gene;
(c) a polynucleotide sequence that hybridizes with the polynucleotide sequence in (a) or (b) under stringent hybridization conditions, wherein the mutation of said polynucleotide sequence endows the maize with the increased root growth angle and lodging resistance;
(d) a polynucleotide sequence that has at least 95% or greater identity to the polynucleotide sequence as set forth in any of (a)-(c); or
(e) a polynucleotide sequence that is complementary to the sequence as set forth in any of (a)-(d).

Optionally, the mutant sequence of the gene promoter refers to the mutant sequence derived by a mutation of the nucleotide sequence of the promoter, wherein said mutation can reduce the transcriptional function of the corresponding promoter, thereby decreasing the expression of the gene driven by this promoter. Preferably, the mutation occurs in a conserved sequence region of the promoter.

Optionally, the mutant sequence of the gene or of the promoter is derived through the mutation comprising substitution, deletion, and/or addition of one or more nucleotides in the nucleotide sequence of the gene or promoter.

Specifically, the mutation can be achieved through physical mutagenesis, chemical mutagenesis, or gene editing. Physical mutagenesis includes but is not limited to radiation mutagenesis, space breeding and the like; methods for chemical mutagenesis include inducing mutagenesis with the treatment of EMS or other mutagenic agents; and methods for gene editing include but are not limited to ZFN, TALEN, and/or CRISPR/Cas or the like.

It is known to those skilled in the art that the main principle of the CRISPR/Cas gene editing system or method is to find a site for gene editing in a host genome through a nucleic acid fragment called guide-RNA (gRNA), i.e., targeting a DNA sequence, and then to cut the DNA through a Cas protein. In the present application, the Cas protein includes but is not limited to Cas9, Cas12, Cas12a, Cas12j, Cas12e, Cas13, and/or Cas14, or other proteins.

Optionally, when the gene editing system used is CRISPR/Cas9, the target sequence used in said CRISPR/Cas9 method to obtain the gene mutant sequence is selected from one of the following sequences:
(a) a sequence being a fragment of a nucleotide sequence as set forth in SEQ ID No: 1, 3, 5, 9, or 10 that conforms to a sequence arrangement rule of 5′-Nx-NGG-3′, wherein N represents any of A, G, C, and T, 14<X<30, X is an integer, and Nx represents X consecutive nucleotides; or
(b) a nucleotide sequence that is complementary to the polynucleotide sequence as set forth in (a).

An embodiment of the present application further provides mutants of the lodging-resistant genes ZmYUC2 and ZmYUC4. Specifically, the two types of mutations in the ZmYUC2 gene include a deletion of 1288 bp-1292 bp (ATTGC) downstream of the start codon (ATG) in the genomic DNA sequence and an insertion of an A base between 1285 bp and 1286 bp; and the two types of mutations in the ZmYUC4 gene include a deletion of the base (A) at 254 bp or 255 bp downstream as well as a deletion of the base (G) at 937 bp or 938 bp downstream, a deletion of 253 bp-267 bp (GAAGACTACCCGGAG) and a deletion of 936 bp-937 bp (CG) downstream of the start codon (ATG) in the genomic DNA sequence.

Optionally, the nucleotide sequence of the gene mutant provided by the embodiment of the present application is as set forth in any of SEQ ID Nos: 11-14.

An embodiment of the present application further provides a method for enhancing lodging resistance of maize by decreasing or inhibiting a normal expression or protein function of a lodging-related gene, wherein the polynucleotide sequence of said gene is selected from one of the following sequences:
(a) a polynucleotide sequence as set forth in SEQ ID No: 1, 3, 5, 9, or 10;
(b) a polynucleotide sequence as set forth in SEQ ID No: 2, 4, or 6, as an encoded amino acid sequence of said gene;
(c) a polynucleotide sequence that hybridizes with the polynucleotide sequence in (a) or (b) under stringent hybridization conditions, inhibiting the expression of said polynucleotide sequence endowing the maize with an increased brace root growth angle and lodging resistance;
(d) a polynucleotide sequence that has at least 95% or greater identity to the polynucleotide sequence as set forth in any of (a)-(c), inhibiting the expression of said polynucleotide sequence endowing the maize with the increased brace root growth angle and lodging resistance; or
(e) a polynucleotide sequence that is complementary to the sequence as set forth in any of (a)-(d).

Optionally, in a method for enhancing the lodging resistance of maize in an embodiment of the present application, decreasing or inhibiting the normal expression or protein function of the lodging-related gene is achieved through RNA interference (i.e. RNAi) and/or mutation. It is known to those skilled in the art that RNAi technology is a conventional technology in the art, which allows for specific binding to a homologous region of an mRNA of a target gene by means of a small interfering RNA (siRNA) or double-strand RNA (dsRNA) of 21-23 bp, enabling mRNA degradation and thus inhibiting gene expression.

Optionally, in the present application, the expression(s) of the lodging-related gene(s) ZmYUC2 and/or ZmYUC4 can be inhibited by RNAi, thereby affecting gene activity and endowing maize plants with a phenotype of increased brace root angle and lodging resistance.

Optionally, in a method for enhancing the lodging resistance of maize in an embodiment of the present application, decreasing or inhibiting the normal expression or protein function of the lodging-related gene is achieved through mutation. The mutation includes substitution, deletion, and/or addition of one or more nucleotides in the nucleotide sequence or promoter sequence of the gene, thereby endowing a plant containing the mutation with an increased brace root angle and thus enhanced lodging resistance.

Optionally, the mutation is achieved by means including but not limited to physical mutagenesis, chemical mutagenesis, and gene editing. Physical mutagenesis includes but is not limited to radiation mutagenesis and space breeding; methods for chemical mutagenesis include inducing mutagenesis with the treatment of EMS or other mutagenic agents; and methods for gene editing include but are not limited to ZFN, TALEN, and/or CRISPR/Cas or the like.

Optionally, in a method for enhancing the lodging resistance of maize in an embodiment of the present application, when the gene editing system CRISPR/Cas9 is used to edit the gene or promoter, the target sequence used in the said CRISPR/Cas9 method is selected from one of the following sequences:
(a) a sequence being a fragment of a nucleotide sequence as set forth in SEQ ID No: 1, 3, 5, 7, 8, 9, or 10 that conforms to a sequence arrangement rule of 5′-Nx-NGG-3′, wherein N represents any of A, G, C, and T, 14<X<30, X is an integer, and Nx represents X consecutive nucleotides; or
(b) a nucleotide sequence that is complementary to the polynucleotide sequence as set forth in (a).

Optionally, a nucleotide sequence after the gene mutation is as set forth in any of SEQ ID Nos: 11-14.

An embodiment of the present application further provides a plant cell, tissue, organ or product that is not used as a reproductive material, wherein said plant cell, tissue, organ or product comprises the mutant sequence as defined in any of the embodiments of the present application.

An embodiment of the present application further provides application of the gene, the expression cassette, recombinant vector or cell, the method as defined in any of the embodiments of the present invention, and a mutant material or transformation event derived therefrom in breeding.

Optionally, the application in breeding refers to the method for enhancing the lodging resistance of maize plants by RNAi, gene mutation, promoter mutation, and/or hybridization with mutant materials.

Optionally, an embodiment of the present invention describes application of the promoter expressed in a local quiescent center and root cap tissue at the tip of root in regulating gene-specific expression in the quiescent center and root cap at the tip of root.

Optionally, an embodiment of the present invention describes application of the promoter expressed in the local root cap tissue at the tip of root in regulating gene-specific expression in the root cap at the tip of root.

Optionally, a method for improving maize root growth angle and lodging resistance described in an embodiment of the present invention involves mutation of the genes ZmYUC2 and ZmYUC4, knocking down for their expressions, or altering of their tissue-specific expressions.

The beneficial effects of the embodiments of the present application are: the lodging-related genes ZmYUC2 and/or ZmYUC4 provided by the embodiments of the present application can regulate auxin synthesis in the local tip of maize root to regulate the gravitropism of maize roots, thereby regulating the maize brace root angle and enhancing the lodging resistance of maize plants, without adverse effects on other agronomic traits. The lodging-related genes, mutants, and their application methods provided by the embodiments of the present application are of great significance for breeding of maize with enhanced lodging resistance.

Definitions of some terms involved in present application:
The term “stringent hybridization conditions” in the present application refers to conditions of low ionic strength and high temperature known in the art. Generally, under stringent conditions, the extent of hybridization of a probe to its target sequence is higher than that to other sequences (e.g., at least 2 times above background). Stringent hybridization conditions are sequence-dependent and vary under different environmental conditions; and longer sequences hybridize specifically at higher temperature. By controlling the stringency of the hybridization or washing conditions, a target sequence 100% complementary to the probe can be identified. More specifically, the stringent conditions are usually chosen to be about 5-10° C. below the thermal melting point (Tm) of the specific sequence at a defined ionic strength and pH. Tm is the temperature at which 50% of the probe complementary to a target hybridizes to the target sequence at equilibrium. The stringent conditions may include the following: the salt concentration is less than about 1.0 M sodium ion concentration at pH 7.0 to 8.3, typically about 0.01 to 1.0 M sodium ion concentration, and the temperature is at least about 30° C. for short probes (including but not limited to 10 to 50 nucleotides), and at least about 60° C. for long probes (including but not limited to greater than 50 nucleotides). The stringent conditions can also be achieved by adding a destabilizing agent such as formamide. For selective or specific hybridization, a positive signal can be at least twice the background hybridization, and in some cases, 10 times the background hybridization. Exemplary stringent hybridization conditions can be as follows: with 50% formamide, 5×SSC, and 1% SDS, incubating at 42° C.; or with 5×SSC and 1% SDS, incubating at 65° C., washing in 0.2×SSC, and washing in 0.1% SDS at 65° C. The washing can be carried out for 5, 15, 30, 60, 120 minutes, or longer.

The term “radicle” refers to the root directly produced by the embryonic tissue after the germination of the maize seed, including a primary root and seminal roots.

The term “primary root” refers to the first root that grows out of the germinating seed.

The term “seminal roots” refers to several roots that grow out of the initial embryo.

The term “nodal roots” refers to roots inserted to the nodes of the maize stem, including crown roots and brace roots.

The term “crown roots” refers to the nodal roots inserted to the underground nodes of maize. The term “brace roots”, also known as “aerial roots”, refers to the nodal roots inserted to the above-ground nodes of maize. The term “gene” in the present application is defined as a genetic unit including one or more polynucleotides, occupying a specific position on a chromosome or plasmid, and containing genetic instructions for specific characteristics or traits in an organism.

The term “RNA interference” (RNAi) refers to a gene-blocking technology involving the process by which double-stranded RNA (dsRNA) molecules block the expression of specific genes at the mRNA level or silence them, namely, sequence-specific post-transcriptional gene silencing (PTGS).

### BRIEF DESCRIPTION OF THE DRAWINGS

FIG. 1 is a graph showing a phylogenetic tree of maize and Arabidopsis YUC proteins, with arrows indicating proteins encoded by ZmYUC2 and ZmYUC4 genes.

FIG. 2 is a graph showing the RT-qPCR analysis of ZmYUC2 and ZmYUC4 genes, with results showing that ZmYUC2 and ZmYUC4 are mainly expressed in maize root tissues.

FIG. 3 is a graph showing in situ hybridization analysis, where ZmYUC2 is mainly expressed in the quiescent center and near the root cap at the tip of root, while ZmYUC4 is mainly expressed in the root cap area at the tip of root; and the tissues used are the tips of maize brace roots.

FIG. 4 is a graph showing CRISPR/Cas9 gene editing and mutant analysis. A shows the design of ZmYUC2 and ZmYUC4 gene-edited target sites; and B shows the genotyping identification of Zmyuc2 and Zmyuc4 single mutants and double mutants.

FIG. 5 is a graph showing the alignment analysis between amino acid sequences of different Zmyuc2 mutants and wild-type amino acid sequences.

FIG. 6 is a graph showing the alignment analysis between amino acid sequences of different Zmyuc4 mutants and wild-type amino acid sequences.

FIG. 7 is a graph showing the phenotypic analysis of brace root angles in Zmyuc2 and Zmyuc4 gene-edited mutants, where (A) shows field root phenotypes of wild type (WT) and mutants at the silking stage, with a white scale of 15 cm. (B-E) show the analyses of the number of aboveground brace root (BR) whorls (B), the number of top BR roots (C), BR diameter (D), and BR growth angle (E) of WT and various Zmyuc mutants in the field at the silking stage, with n>20. (F) shows X-ray CT images of WT and various Zmyuc mutants grown to the V6 stage in soil, with red arrows indicating above-ground stem roots and a yellow scale of 5 cm. (G-H) shows the number (G) and angle (H) of stem roots measured from the X-ray CT images, with n>10.

FIG. 8 is a graph showing that field density planting experiments indicate that under high-density planting conditions, the root lodging resistance of Zmyuc2/Zmyuc4 double mutants is significantly enhanced compared to wild-type controls. A shows the comparison of root lodging resistance between Zmyuc2/Zmyuc4 double mutants and wild-type controls at the tasseling stage in Langfang in 2021; and the horizontal axis shows the angle between the stem and the vertical line, and the vertical axis shows the force used to push the stem to a certain angle. B shows images of root lodging under high density (135,000 plants/ha), taken by a drone equipped with a camera with a 1/1.3 inch (48 million pixel) image sensor. C shows the analysis of root lodging of WT, Zmyuc2, Zmyuc4 single mutants, and double mutants under high density. Level 1 indicates lodging severity≤30°. Level 2 indicates 30°<lodging severity≤60°. Level 3 indicates lodging severity>60°.

FIG. 9 is a graph showing the field phenotype observations, indicating that single and double mutants of Zmyuc2 and Zmyuc4 do not cause significant changes in plant height, leaf morphology, and other plant architecture-related traits.

FIG. 10 is a graph showing the field phenotype observations, indicating that single and double mutants of Zmyuc2 and Zmyuc4 do not cause significant changes in ear size, grain, and yield-related traits.

FIG. 11 is a graph showing the impact of ZmYUC2 and ZmYUC4 gene mutations on local auxin content and gravity response in the root tip. A and C show the comparison (A) and statistics (C) of RFP fluorescence intensity in the root tips growing along the gravity direction in control CK, and various Zmyuc2 and Zmyuc4 mutants. B shows the comparison of RFP fluorescence intensity in root tips growing perpendicular to the gravity direction in control CK, and various Zmyuc2 and Zmyuc4 mutants. D shows the statistics of RFP fluorescence intensity in the upper and lower epidermis (indicated by arrows in panel B) of root tips growing perpendicular to the gravity direction in control CK, and various Zmyuc2 and Zmyuc4 mutants. Overall, changes in RFP fluorescence intensity indicate that auxin content in the root cap is reduced in Zmyuc2 and Zmyuc4 mutants, affecting the distribution of auxin content in the root tip after gravity stimulation.

FIG. 12 is a graph showing that the ZmYUC2 gene has been subjected to significantly artificial selection during modern maize breeding in China. A shows the comparative analysis of brace root phenotypes among different generations of the Chinese Huangzaosi subgroup; ** indicates highly significant differences; and ns indicates no significant difference. B shows the selection signal profile of the ZmYUC2 gene region (XP-CLR method); the “<” symbol indicates the position of the ZmYUC2 gene; the top horizontal dashed line indicates the significance threshold of the top 2% selection signals across the genome; the upper part of the figure shows the selection signals among different generations of Chinese inbred lines, and the lower part shows the selection signals among different generations of the Huangzaosi subgroup.

### DETAILED DESCRIPTION OF THE DRAWINGS AND THE PRESENTLY PREFERRED EMBODIMENTS

To facilitate understanding of the present application, a more comprehensive description of the present application is provided below. However, the present application may be implemented in many different forms and is not limited to the embodiments described herein. On the contrary, these embodiments are provided for the purpose of more thorough and comprehensive understanding of the disclosure of the embodiments of the present application.

The inbred lines used in the following embodiments can be found on the Chinese Crop Germplasm Resources Information System (CGRIS), where relevant information can be accessed, and corresponding seeds can be requested.

### Embodiment 1: Gene Expression Analysis Indicating Specific Expression of ZmYUC2 and ZmYUC4 Mainly in Maize Root Tissues

### 1. Homologous Protein Analysis Identified 14 YUC Protein-Encoding Genes in the Maize Genome.

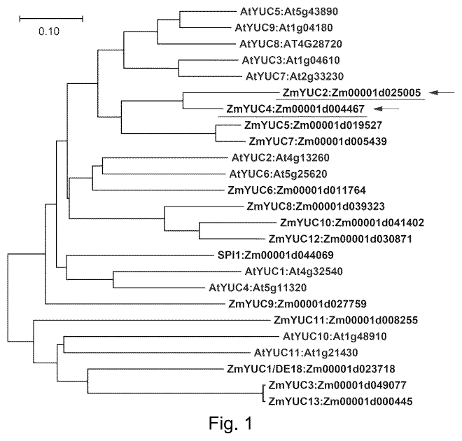

The inventors searched the maize sequence database B73 AGPv4 in the Gramene database (http://ensembl.gramene.org/Zea_mays/Info/Index) with BLASTP (E-value set to 1e-10, other parameters set to default) using the sequences of reported Arabidopsis YUC proteins (YUC3, YUC5, YUC7, YUC8, and YUC9) as query sequences (Chen et al., 2014), and identified 14 YUC homologous genes (FIG. 1), named: SPI1, DE18, ZmYUC2, ZmYUC3, ZmYUC4, ZmYUC5, ZmYUC6, ZmYUC7, ZmYUC8, ZmYUC9, ZmYUC10, ZmYUC11, ZmYUC12, and ZmYUC13 (FIG. 1).

Further analysis revealed that the ZmYUC4 gene (genomic nucleotide sequence as set forth in SEQ ID No: 10) had only one transcript (nucleotide sequence as set forth in SEQ ID No: 5, amino acid sequence as set forth in SEQ ID No:6), whereas the ZmYUC2 gene (genomic DNA sequence as set forth in SEQ ID No: 9) had two transcripts: ZmYUC2-T001 (nucleotide sequence as set forth in SEQ ID No: 1, amino acid sequence as set forth in SEQ ID No:2) and ZmYUC2-T002 (nucleotide sequence as set forth in SEQ ID No: 3, amino acid sequence as set forth in SEQ ID No: 2). Transcriptome data analysis indicated that ZmYUC2-T002 (SEQ ID No: 3) was the dominant transcript of the ZmYUC2 gene, which was 102 bp longer than ZmYUC2-T001 (SEQ ID No: 1). The two transcripts differ from each other only after the 1030 bp, and both encoded proteins were expected to perform the corresponding functions. Subsequent analyses were based on primers and probes designed for the sequence before the 1030 bp position.

2. Gene Expression Analysis Indicated ZmYUC2 and ZmYUC4 were Mainly Expressed in Maize Root Tissues:

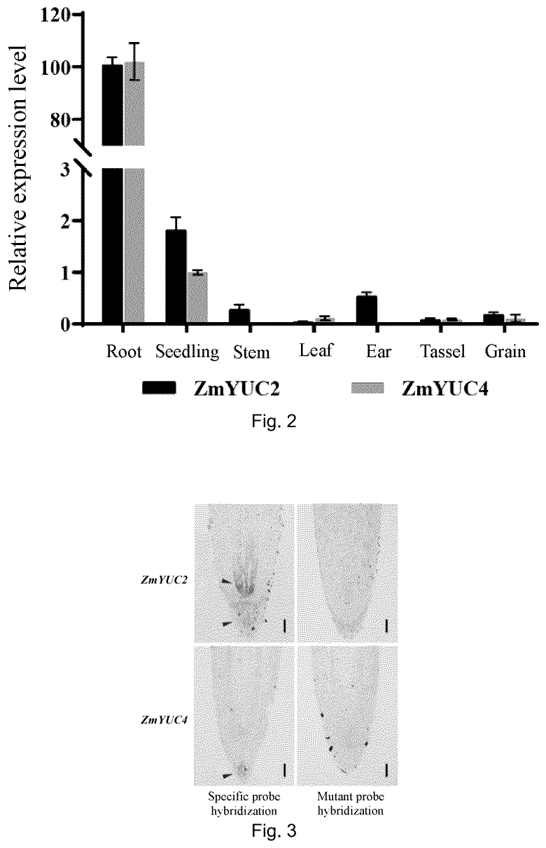

with the published gene expression data across all growth stages of maize (https://www.maizegdb.org/), a heat map of ZmYUCs gene expression was created. Analysis revealed that seven ZmYUC genes were expressed in maize root-related tissues, with ZmYUC2 and ZmYUC4 showing high root system-specific expression (predominantly expressed in roots, with low or no expression in other tissues, FIG. 2). To validate the tissue-specific expression of ZmYUC2 and ZmYUC4, the inventors performed qRT-PCR analysis on roots and above-ground parts of B73 inbred line seedlings at the V1 stage, stems (10th node), leaves (topmost expanded leaf), young ears, and young tassels at the V13 stage, and grains 15 days post-pollination. Results indicated that ZmYUC2 and ZmYUC4 were indeed specifically expressed in maize root tissues (FIG. 2), suggesting that ZmYUC2 and ZmYUC4 might play important roles in regulating maize root development.

### Embodiment 2: In Situ Hybridization Showing Expression of ZmYUC2 Mainly in the Quiescent Center and Root Cap of Maize Root Tips and Expression of ZmYUC4 Mainly in the Root Cap of Maize Root Tips

To determine the specific tissue expression sites of ZmYUC2 and ZmYUC4, the inventors designed specific probes for these two genes and conducted in situ hybridization experiments on young brace root tips of the B73 inbred line. Results showed (FIG. 3) that ZmYUC2 was mainly expressed in the quiescent center and root cap at the maize root tips, while ZmYUC4 is mainly expressed in the root cap at the root tips. The quiescent center is an important tissue that controls the differentiation of surrounding stem cells and maintains the activity of the root apical meristem. The root cap is a key tissue for gravity signal perception in plant root tips. These results suggest that ZmYUC2 and ZmYUC4 may play important roles in the regulation of gravitropism in maize roots.

### Embodiment 3: Zmyuc4 Single Mutant and Zmyuc2/Zmyuc4 Double Mutants to Increase Root Growth Angle and Lodging Resistance in Maize without Adverse Effects

1. The Brace Root Angle and Surface Root Coverage of the Zmyuc2/Zmyuc4 Double Mutants were Significantly Increased Compared to the Wild Type.

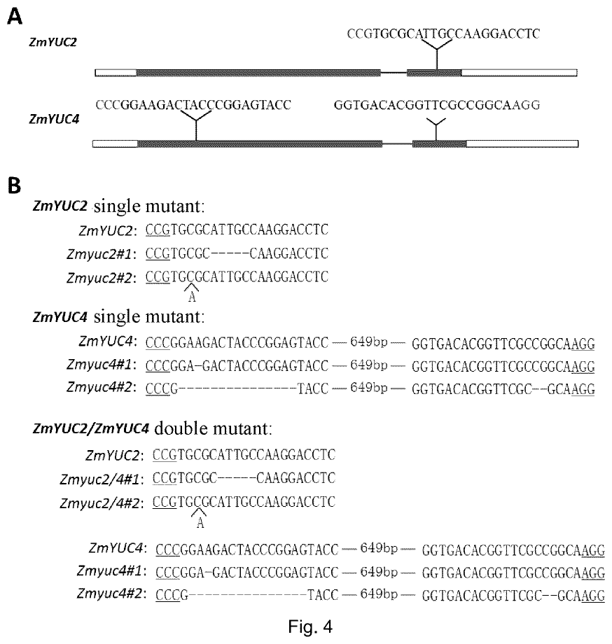

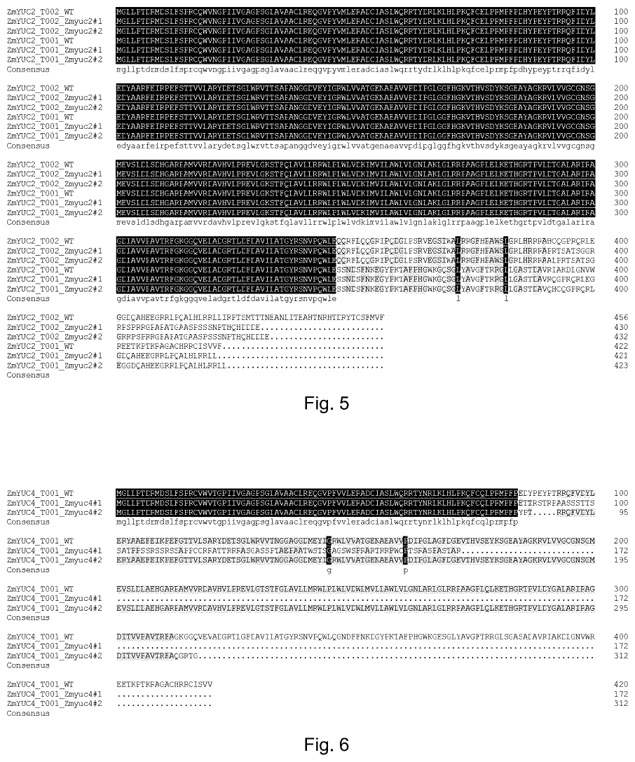

To determine the biological functions of ZmYUC2 and ZmYUC4, the inventors constructed CRISPR/Cas9 gene editing vectors for these genes (FIG. 4A) and genetically transformed the maize inbred line ZC01. By performing PCR detection and separating the transgenic vectors in T1 generation transgenic materials, two independent lines of Zmyuc2 and Zmyuc4 single mutants, as well as Zmyuc2/Zmyuc4 double mutants, were obtained (FIG. 4B), named Zmyuc2 #1, Zmyuc2 #2, Zmyuc4 #1, Zmyuc4 #2, Zmyuc2/4 #1, and Zmyuc2/4 #2. Analysis revealed that the two types of mutations in the ZmYUC2 gene included a deletion of 1288 bp-1292 bp (ATTGC) downstream of the start codon (ATG) (SEQ ID No:11) in the genomic DNA sequence and an insertion of an A base between 1285 bp and 1286 bp (SEQ ID No: 12); and the two types of mutations in the ZmYUC4 gene were a deletion of the base (A) at 255 bp downstream as well as a deletion of the base (G) at 938 bp downstream (SEQ ID No: 13), and a deletion of 253 bp-267 bp (GAAGACTACCCGGAG) and a deletion of 936 bp-937 bp (CG) downstream of the start codon (ATG) (SEQ ID No:14). Amino acid sequence analysis indicated that these insertions and deletions of bases caused premature termination or frame shift mutations in the ZmYUC2 and/or ZmYUC4 genes in the mutants. Both transcripts of the ZmYUC2 gene were mutated. The resulting encoded protein amino acid sequences and their alignments with the wild type were shown in FIG. 5 for ZmYUC2 and FIG. 6 for ZmYUC4 mutants.

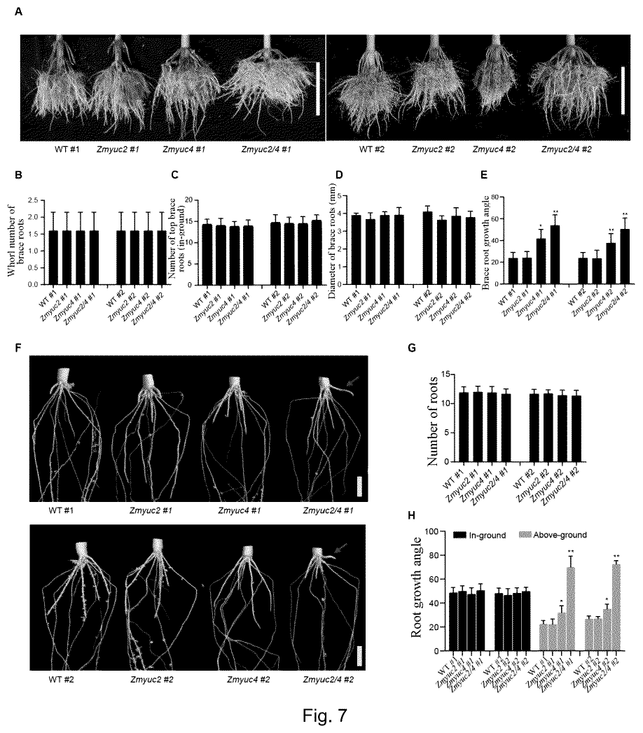

Field phenotype analysis in the summer of 2021 at the Langfang experimental station in Hebei showed that the brace root angles and surface root coverage resulting from Zmyuc2/Zmyuc4 double mutants were significantly larger than those of the wild type (FIGS. 7A and 7E). The phenotype of increased brace root angle by Zmyuc21 Zmyuc4 double mutants was confirmed again during the winter of 2021 at the Hainan experimental base. By comparing the phenotypes of the single and double mutants, X-ray CT further revealed that while Zmyuc2 single mutants did not significantly differ from wild type in brace root angles, Zmyuc4 single mutants had larger angles than wild type but smaller than the double mutants (FIGS. 7F and 7H). This suggested functional redundancy between ZmYUC2 and ZmYUC4 in regulating the brace root angles. Additional analyses showed no significant differences in the number of brace and crown roots between mutants and wild type (FIGS. 7B-D and 7G), indicating a specific regulatory role of ZmYUC2 and ZmYUC4 in brace root angles.

### 2. Significantly Enhanced Lodging Resistance by Zmyuc4 Single Mutant and Zmyuc2/Zmyuc4 Double Mutants

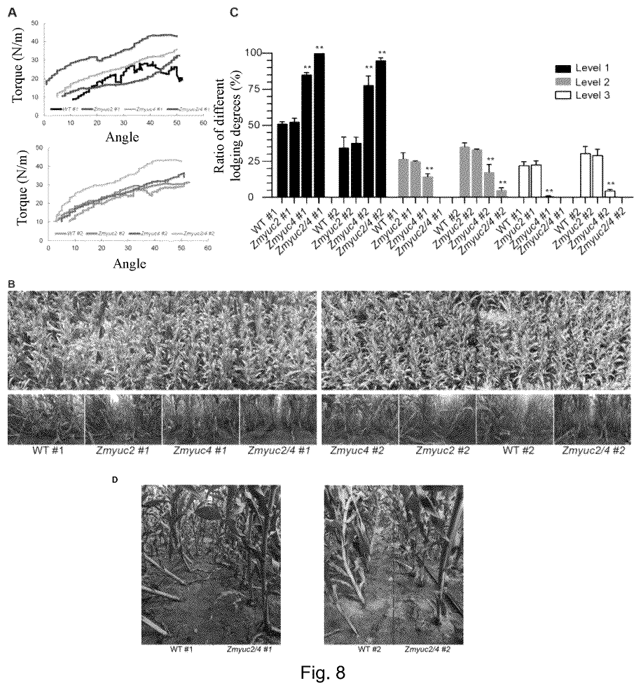

To explore the role of ZmYUC2 and ZmYUC4 in maize lodging resistance, a dynamic root lodging tester was used to measure the root lodging force for Zmyuc2/Zmyuc4 double mutants and wild type controls at the tasseling stage (2021 in Langfang). It was found that the force required to push the stems of Zmyuc4 single mutants and Zmyuc2/Zmyuc4 double mutants to the same angle (deviating from vertical) was significantly greater than that for wild type, with the double mutants requiring the most force (FIG. 8A). It is particularly noteworthy that during a storm in early July 2021 in Langfang, the ZC01 genetic materials (background of ZmYUC2 and ZmYUC4 gene-edited receptor materials) experienced varying degrees of lodging in the field. However, the lodging rate of the two Zmyuc2/Zmyuc4 double mutants was significantly reduced compared to the wild-type materials (FIG. 8D). High-density planting is an effective method to increase maize yield, but it also increases the risk of lodging, which can reduce maize production. To evaluate the root lodging resistance of Zmyuc mutants at different planting densities, we conducted a density experiment in Langfang in 2022. The experiment included three densities: D1 (45,000 plants per hectare), D2 (90.000 plants per hectare), and D3 (135,000 plants per hectare), with three replicates for each density. Lodging severity was recorded during the silking stage. The lodging severity was classified into three levels based on the deviation angle: Level 1 (less than 30°), Level 2 (30°-60°), and Level 3 (greater than 60°). The determined lodging severity between Zmyuc mutants and wild type (WT) showed that lodging rates increased with planting density in wild type, Zmyuc2, and Zmyuc4, but Zmyuc2/Zmyuc4 double mutants remained upright across the three densities (FIGS. 8B and 8C). This suggests that Zmyuc4 single mutants and Zmyuc2/Zmyuc4 double mutants can be used for breeding maize with improved lodging resistance.

### 3. Both Single and Double Mutations of ZmYUC2 and ZmYUC4 Genes Showing No Adverse Effects on the Aboveground Agronomic Traits of Maize Plants

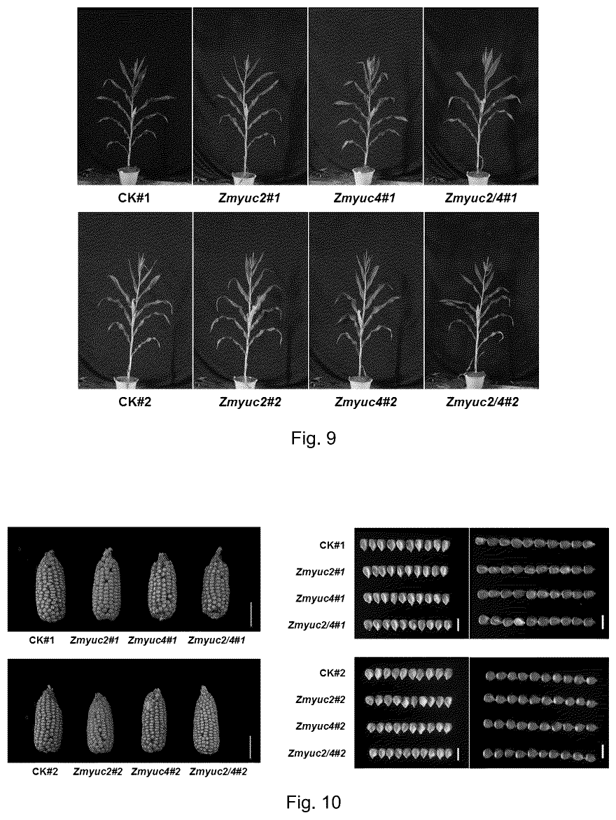

Phenotypic observations from the 2021 growing seasons in Langfang and Hainan indicated no significant differences in plant height, leaf architecture, inflorescence traits (FIG. 9), grain, and ear yield (FIG. 10) between Zmyuc2 and Zmyuc4 single mutants, Zmyuc2/4 double mutants, and wild type controls. These results suggest that modifying root architecture without altering overall root biomass or other agronomic traits is a key method for breeding high-yield, lodging-resistant maize varieties. Thus, ZmYUC2 and ZmYUC4 have great potential for application in breeding programs aimed at improving maize lodging resistance and yield.

### Embodiment 4: ZmYUC2 and ZmYUC4 Genes to Regulate Root Gravitropism by Modulating Local Auxin Content and Distribution in Root Tips

According to a report (Gallavotti A, Yang Y, Schmidt R J, et al. The relationship between auxin transport and maize branching[J]. Plant physiology, 2008, 147 (4): 1913-1923), the DR5 promoter, created using 9 tandem repeats of the auxin-responsive element (AuxRE), drives the expression of reporter genes. Methods such as DR5::RFP can effectively reflect the accumulation levels of auxin within plant tissues.

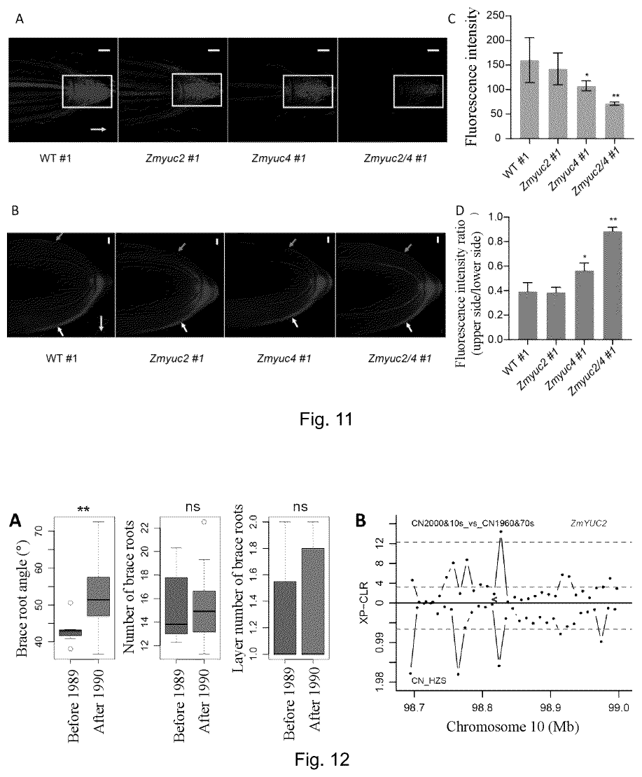

To determine whether ZmYUC2 and ZmYUC4 influence maize root gravitropism through auxin abundance regulation, the inventors crossed DR5::RFP transgenic materials with Zmyuc2 and Zmyuc4 gene-edited mutant lines. They created the genetic materials Zmyuc2/DR5::RFP, Zmyuc4/DR5::RFP, and Zmyuc2/Zmyuc4/DR5::RFP, incorporating DR5::RFP into the Zmyuc2, Zmyuc4 single mutants, and the Zmyuc2/Zmyuc4 double mutants. Roots were cultured along the gravity direction, and the auxin content was assessed by observing the fluorescence intensity in the root tip region (gravitropic sensing tissues). The results (FIGS. 11A and 11C) showed that auxin concentration in the root tip columella cells significantly decreased following mutations in ZmYUC2 and ZmYUC4 genes. The auxin content reduction was more severe in the Zmyuc4 single mutant compared to the Zmyuc2 single mutant, and the most severe reduction was observed in the Zmyuc2/Zmyuc4 double mutants. These findings indicate that ZmYUC2 and ZmYUC4 genes regulate local auxin content in root tips, confirming the functional redundancy between ZmYUC2 and ZmYUC4 genes.

Further, roots were cultured along the vertical gravity direction to simulate gravitational stimulation. Observations revealed that in each material (wild type, Zmyuc2 single mutant, Zmyuc4 single mutant, and Zmyuc2/Zmyuc4 double mutants), the auxin content in the elongation zone of the root was higher on the lower (gravitropic) side compared to the upper (anti-gravitropic) side (FIGS. 11B and 11D). However, comparing different materials, it was found that in Zmyuc2, Zmyuc4 single mutants, and Zmyuc2/Zmyuc4 double mutants, the auxin content in the elongation zone of the root toward both the gravity and anti-gravity sides was significantly lower compared to the corresponding parts in the wild-type (FIGS. 11B and 11D). Particularly, the Zmyuc2/Zmyuc4 double mutants exhibited the most significant reduction in auxin content. Further comparisons revealed that the reduction in auxin content on the lower side compared to the upper side in the root elongation zone was more pronounced in Zmyuc2, Zmyuc4 single mutants, and Zmyuc2/Zmyuc4 double mutants than in the wild-type maize plants. The differential auxin content between the lower and upper sides of the root elongation zone after gravity stimulation is the direct cause of root gravitropic response and bending towards gravity. Therefore, it can be concluded that the ZmYUC2 and ZmYUC4 genes regulate root gravitropism by controlling the local auxin content and distribution at the tip of root. The sensitivity and speed of root response to gravity determine the rate at which roots bend towards the ground, thereby influencing the size of the root-shoot angle. Therefore, ZmYUC2 and ZmYUC4 are likely involved in regulating maize root gravitropism by controlling the synthesis and content of auxin at the tip of root, thereby influencing the root gravitropic response and consequently, the root-shoot angle in maize.

### Embodiment 5: ZmYUC2 Gene Undergoing Strong Artificial Selection During Modern Maize Breeding

The inventors collected 350 maize breeding materials from China and the USA from different eras. In the winter of 2021, phenotypic measurements of brace root angle, number of layers, and number of topmost brace roots were conducted on these 350 maize inbred line materials at the Ledong experimental base in Hainan. By comparing the changes in traits among materials from different years, they found that in the Chinese-specific Huangzaosi subgroup materials, the angle of brace roots showed an overall trend of increasing from smaller angles in earlier materials to larger angles in more recent materials during breeding (significantly smaller in early materials compared to modern ones), while the number and layers of brace roots did not show significant changes (FIG. 12A). This suggests that the brace root angle is a key breeding target in the Huangzaosi subgroup. Further analysis involved re-sequencing the 350 materials at an average depth of 13.4× and using the XP-CLR method for genome-wide selection scans, identifying 1,888 significant selective sweeps. Among them, the ZmYUC2 gene fell within one of these significant selection regions (FIG. 12B). Deeper analysis revealed that the ZmYUC2 gene underwent significant artificial selection during the breeding process from early (1960s and 1970s) to modern times (2000s to present), particularly in the Chinese-specific Huangzaosi subgroup. This suggests that the ZmYUC2 gene plays an important role in the breeding improvement of Chinese maize germplasm, especially within the Huangzaosi subgroup. Improving lodging resistance has always been a primary breeding goal in maize improvement, especially in the Huangzaosi subgroup, which also reflects the importance of the selection and application of the ZmYUC2 gene in maize breeding for lodging resistance.

The embodiments described above only provide specific and detailed descriptions of several embodiments of the present invention, and should not be construed to limit the patent scope of the present invention. It should be noted that several variations and improvements can be made by those of ordinary skill in the art without departing from the concept of the present invention, and these variations and improvements shall fall within the protection scope of the present invention.

1. A gene mutant sequence that is derived from mutation of a gene nucleotide sequence, and generates a phenotype of an increased brace root growth angle and lodging resistance in maize plants containing the gene mutant sequence, wherein said gene nucleotide sequence is selected from one of the following sequences:
(a) a polynucleotide sequence as set forth in SEQ ID No: 1, 3, 5, 9, or 10;
(b) a polynucleotide sequence as set forth in SEQ ID No: 2, 4, or 6, as an encoded amino acid sequence of said gene;
(c) a polynucleotide sequence that hybridizes with the polynucleotide sequence in (a) or (b) under stringent hybridization conditions, wherein the mutation of said polynucleotide sequence endows the maize with the increased brace root growth angle and lodging resistance;
(d) a polynucleotide sequence that has at least 95% or greater identity to the polynucleotide sequence as set forth in any of (a)-(c); or
(e) a polynucleotide sequence that is complementary to the sequence as set forth in any of (a)-(d).

2. The gene mutant sequence of claim 1, wherein said gene mutant sequence is derived through the mutation comprising substitution, deletion, and/or addition of one or more nucleotides in the nucleotide sequence of said gene.

3. The gene mutant sequence of claim 2, wherein said mutation is generated by techniques comprising physical mutagenesis, chemical mutagenesis, ZFN, TALEN, and/or CRISPR/Cas gene editing.

4. The gene mutant sequence of claim 3, wherein said CRISPR/Cas gene editing involves CRISPR/Cas9, in which a target sequence is selected from one of the following sequences:
(a) a sequence being a fragment of a nucleotide sequence as set forth in SEQ ID No: 1, 3, 5, 9, or 10 that conforms to a sequence arrangement rule of 5′-Nx-NGG-3′, wherein N represents any of A, G, C, and T, 14<X<30, X is an integer, and Nx represents X consecutive nucleotides; or
(b) a nucleotide sequence that is complementary to the polynucleotide sequence as set forth in (a).

5. The gene mutant sequence of claim 1, wherein said gene mutant sequence is as set forth in any of SEQ ID Nos: 11-14.

6. A method for enhancing lodging resistance in maize by decreasing or inhibiting a normal expression or protein function of a lodging-related gene, wherein the polynucleotide sequence of said gene is selected from one of the following sequences:
(a) a polynucleotide sequence as set forth in SEQ ID No: 1, 3, 5, 9, or 10;
(b) a polynucleotide sequence as set forth in SEQ ID No: 2, 4, or 6, as an encoded amino acid sequence of said gene;
(c) a polynucleotide sequence that hybridizes with the polynucleotide sequence in (a) or (b) under stringent hybridization conditions, inhibiting the expression of said polynucleotide sequence endowing the maize with an increased brace root growth angle and lodging resistance;
(d) a polynucleotide sequence that has at least 95% or greater identity to the polynucleotide sequence as set forth in any of (a)-(c), inhibiting the expression of said polynucleotide sequence endowing the maize with the increased brace root growth angle and lodging resistance; or
(e) a polynucleotide sequence that is complementary to the sequence as set forth in any of (a)-(d).

7. The method of claim 6, wherein decreasing or inhibiting the normal expression or protein function of the lodging-related gene is achieved by RNA interference and/or mutation.

8. The method of claim 7, wherein decreasing or inhibiting gene expression or gene mutation comprises substitution, deletion, and/or addition of one or more nucleotides in a nucleotide sequence of said gene or a gene promoter.

9. The method of claim 6, wherein inhibiting the normal expression of the lodging-resistant genes is achieved by means comprising RNAi, physical mutagenesis, chemical mutagenesis, ZFN, TALEN, and/or CRISPR/Cas gene editing.

10. The method of claim 9, wherein said CRISPR/Cas is the CRISPR/Cas9 gene editing method, with a target sequence selected from one of the following sequences:
(a) a sequence being a fragment of a nucleotide sequence as set forth in SEQ ID No: 1, 3, 5, 7, 8, 9, or 10 that conforms to a sequence arrangement rule of 5′-Nx-NGG-3′, wherein N represents any of A, G, C, and T, 14<X<30, X is an integer, and Nx represents X consecutive nucleotides; or
(b) a nucleotide sequence that is complementary to the polynucleotide sequence as set forth in (a).

11. The method of claim 6, wherein a nucleotide sequence after said gene mutation is as set forth in any of SEQ ID Nos: 11-14.
